(19) United States
(12) Patent Application Publication
(10) Pub. No.: US 2019/0267118 A1
(43) Pub. Date: Aug. 29, 2019

(54) PERSON-CENTERED HEALTH RECORD ARCHITECTURE

(71) Applicants: Titus Karl Ludwig SCHLEYER, Indianapolis, IN (US); Zina Ben MILED, Zionsville, IN (US); Zachary Doyle KING, Indianapolis, IN (US); Indiana University Research and Technology Corporation, Indianapolis, IN (US)

(72) Inventors: Zina Ben Miled, Zionsville, IN (US); Titus Karl Ludwig Schleyer, Indianapolis, IN (US); Zachary Doyle King, Chicago, IL (US)

(73) Assignee: Indiana University Research and Technology Corporation, Indianapolis, IN (US)

(21) Appl. No.: 16/349,040

(22) PCT Filed: Nov. 13, 2017
(86) PCT No.: PCT/US2017/061254

§ 371 Date: May 10, 2019

Related U.S. Application Data
(60) Provisional application No. 62/420,281, filed on Nov. 10, 2016.
(60) Provisional application No. 62/530,672, filed on Jul. 10, 2017.

(51) Int. Cl. G16H 10/60; G16H 15/00

(52) U.S. Cl. CPC G16H 10/60; G16H 15/00

(57) ABSTRACT

Methods for providing a health record architecture, comprising linking a patient's health information to relevant health data, automatically determining an applicability of current medical evidence, recommendations, or combinations thereof to the patient, and enriching a health record from external data sources are disclosed. Health record architectures are also disclosed.

### CROSS-REFERENCE TO RELATED APPLICATIONS

This application claims priority to U.S. Provisional Application Ser. No. 62/420,281, filed on Nov. 10, 2016, and to U.S. Provisional Application Ser. No. 62/530,672, filed on Jul. 10, 2017 the entire disclosures of which are each hereby expressly incorporated by reference in their entirety.

### TECHNICAL FIELD

The present disclosure includes matter generally related to patient-centered health record information systems. More specifically, this disclosure includes personalized patient-centered health record information systems and methods.

### BACKGROUND

Recent, significant investments in health information technology (health IT) have resulted in an unprecedented increase in the “digitization” of the health care system in the US. However, the digitization of health care information has not automatically translated into increased utility and usability for patients and providers. An example of the practical limitations common today is the fact that patients often must interact with more than one patient portal, especially when they see clinicians in multiple health systems. This makes it difficult for patients and clinicians to review and synthesize the patient's health information, and act efficiently and effectively on it.

A second limitation in conventional systems and methods is that the health information captured about patients is organized using a one-size-fits-all model. For instance, even in electronic systems male patients are routinely asked whether they are pregnant or not. Thus, health information is not easy for individuals, their families, and care providers to send, receive, find, and use in a manner that is appropriate, secure, timely, and reliable.

Digitization of the health sector emphasizes three main initiatives: 1) migrating legacy ad hoc electronic health records (EHR) into standardized enterprise-level EHR systems, 2) facilitating EHR-to-EHR interoperability and exchanges of health information, and 3) enabling EHR-to-Patient/PHR exchanges of this health information.

The success of the first initiative, the migration to standardized EHR systems, has resulted in 93% of non-Federal acute care hospitals adopting certified EHR systems by 2014 according to the ONC, and 200 million patients in the US expected to be covered by the Epic™ EHR, system once all of its ongoing system deployments are completed.

With regard to the second initiative, EHR-to-EHR interoperability, the federal government launched an unprecedented nationwide effort in 2009 to help evolve a digital, interoperable health care system in the US. This effort resulted in more than 50% of office-based professionals and 80% of hospitals using EHRs through which they are now able to electronically exchange standardized patient information in a basic fashion. A similar state-wide EHR interoperability endeavor is the Indiana Network for Patient Care (INPC), which was started by the Regenstrief Institute in 1998 and is now operated by the Indiana Health Information Exchange. Today, the INPC connects 25,000 physicians, 106 hospitals, 110 clinics, surgery centers and many other health care organizations, and maintains information about over 14 million patients.

In connection with the third initiative, EHR-to-Patient/PHR information exchange, it was estimated that as of 2012, 57% of health care providers had a patient portal solution in place allowing patients to access all or part of their health information. Moreover, in order to investigate the challenges of supporting two-way information exchange between patients and health care providers, the ONC conducted a pilot through the National Association for Trusted Exchange (NATE). This pilot focused on data flow and exchange of information from the provider to the person health record PHR (EHR-to-PHR) and vice-versa (PHR-to-EHR). The NATE pilot bi-directionally connected EHRs to PHRs using Blue Button and HealthVault. Blue Button allows patients to electronically access their own health information from providers such as health plans, pharmacies, and hospitals. HealthVault is a PHR that allows patients to connect to health providers via Blue Button and download their health related information into a cloud-based account.

The three groundbreaking initiatives mentioned above were important enablers to support the difficult “last mile” effort that will deliver much anticipated benefits of PHRs to the patient. Currently several patient-oriented software applications are being offered as solutions to bridge the last mile. However, the limitations of these solutions are still significant enough to have prompted the ONC to articulate its recent 10-year vision. This vision encourages “last mile” solutions that a) enable individuals to manage their own health information, b) share it with their health care providers and c) enable health care providers to individualize diagnosis and therapy and adapt it, as needed, to the patient's condition in real-time by 2025.

Conventional systems, include systems such as HealthVault, Google Health and Indivo-Dossia can be categorized as personal health data-marts. HealthVault provides the ability to organize patient health information (PHI), register medical devices (for acquisition of parameters such as blood pressure and glucose) and aggregate family accounts. Under this model, patients have a “health” account on the cloud that is used to house their records retrieved from different health care providers' EHRs, which are, in the overwhelming number of cases, completely independent of each other.

Google Health was an early PHR that used a model similar to that of HealthVault. It was discontinued in 2011. Indivo is an open source PHR which is supported by several large corporations including WalMart. It allows a limited level of personalization through the selection of apps such as immunization tracker and medication manager. These pioneering tools use a relational model with a focus on collecting data from different sources.

An additional feature of Indivo-Dossia was the ability to automate the process flow between the health service provider and the patient. For instance, a patient may request an examination by using Indivo. The doctor can then review the patient's health profile and update it by entering a diagnosis or ordering a laboratory test. The request for the laboratory test is forwarded to the information management system of the corresponding laboratory. Once the test is completed, the results are entered and the doctor is notified. Indivo uses ontologies to semantically disambiguate concepts that may be expressed differently in the participating sub-systems.

MeTree and Health Heritage are decision support systems. MeTree focuses on collecting and organizing family history in order to help support primary care decision-making. Health Heritage focuses on matching the family history with recent scientific research in order to personalize care and prevention plans.

Given the variety and multitude of these types of patient-focused software applications, some of which have been in existence for nearly a decade, widespread community adoption was lacking. The result of a survey of PHR penetration rates in the US indicated that, in 2010, only 7% of the survey participants had ever used a PHR and less than half of these continued using it. Among veterans, 71% of which utilize the Internet, about 20% reported using a PHR in a 2012 survey.

In an attempt to answer the above question and by using a process of elimination, cost can be excluded since most of the above-mentioned patient-focused software applications are free. Privacy concerns can also be excluded based on the findings of an earlier study. This study indicated that while a reasonable level of privacy is expected, a large percentage of the participants were willing to share their health information. The answer can then hypothetically be attributed to 1) the community interest level or 2) ease of use and the added benefit trade-off. It was found that community interest level can also be excluded. This conclusion is supported with a consensus around the fact that the adoption of PHRs is low in spite of the anticipated benefits. Some of the identified reasons for the low adoption include the need for additional education and training but most importantly for a better PHR model that can leverage technological advances while meeting the needs of patients and health care professionals.

Thus, improved person health record (PHR) systems and methods are needed to address the aforementioned limitations of conventional systems and allow for widespread adoption.

### SUMMARY

According to one embodiment, the present disclosure provides a model for patient-centered health record information systems that is focused on the patient and their conditions, and emphasizes patient-involved health care management. The Personalized Person Health Record (P2HR) is personalized on demand to the conditions of each individual, and organized to facilitate the tracking and review of the patient's conditions by both the patient and their health care providers.

In some embodiments, methods for providing a health record architecture, may include linking a patient's health information to relevant health data, automatically determining an applicability of current medical evidence, recommendations, or combinations thereof to the patient, and enriching a health record from external data sources.

The present disclosure also provides for various health record architecture systems. In some embodiments, the health record architectures may include a display, a processor in communication with an electronic health record database and a non-transitory, computer readable storage medium, the non-transitory, computer readable storage medium having instructions stored therein that cause the processor to: extract and link a patient's health information from an electronic health record to relevant health data, automatically determine the applicability of current medical evidence, recommendations, or combinations thereof to the patient, and enrich the health record.

The present disclosure also provides for non-transitory computer readable storage mediums bearing instructions for providing a health record architecture, the instructions, when executed by a processor in electrical communication with an electronic health care database, cause the processor to perform operations including linking a patient's health information to relevant health data, automatically determining an applicability of current medical evidence, recommendations, or combinations thereof to the patient, and enriching a health record from the electronic health care database.

While multiple embodiments are disclosed, still other embodiments in the present disclosure will become apparent to those skilled in the art from the following detailed description, which shows and describes illustrative embodiments. Accordingly, the drawings and detailed description are to be regarded as illustrative in nature and not restrictive.

### BRIEF DESCRIPTION OF THE DRAWINGS

The above-mentioned and other features of this disclosure and the manner of obtaining them will become more apparent and the disclosure itself will be better understood by reference to the following description of embodiments of the present disclosure taken in conjunction with the accompanying drawings, wherein:

FIGS. 1A and 1B are flow charts for methods for providing a health record architecture according to various embodiments;

FIG. 2 is a conceptual diagram of an exemplary health record architecture according to various embodiments;

FIG. 3 is a conceptual diagram of an exemplary three tier health record architecture according to various embodiments;

FIG. 4 is a conceptual diagram of a personalized person-centered (P2HR) architecture according to various embodiments of the present disclosure;

FIG. 5 is a sample code depiction of primitive objects according to various embodiments; and

FIG. 6 is a sample code depiction of a P2HR data model according to an embodiment.

While the present disclosure is amenable to various modifications and alternative forms, specific embodiments have been shown by way of example in the drawings and are described in detail below. The present disclosure, however, is not to limit the particular embodiments described. On the contrary, the present disclosure is intended to cover all modifications, equivalents, and alternatives falling within the scope of the appended claims.

### DETAILED DESCRIPTION

One of ordinary skill in the art will realize that the embodiments provided can be implemented in hardware, software, firmware, and/or a combination thereof. Programming code according to the embodiments can be implemented in any viable programming language such as C, C++, Python, HTML, XTML, JAVA or any other viable high-level programming language, or a combination of a high-level programming language and a lower level programming language.

Against the backdrop of the limitations discussed in the Background above, the increasing penetration of health IT, coupled with the now commonplace expectation that we can manage almost all aspects of our lives electronically, presents an unprecedented opportunity to shape the health sector via a socially-driven, digital approach for person health record (PHR) management.

The various embodiments described herein present various methods and systems to help overcome the gaps preventing the mainstream adoption of PHRs. Various embodiments include systems and methods that can include the automated generation of a PHR customized to individual patients and their health and disease conditions. Through these various systems and methods, patients will have the opportunity to manage their own health information based on their specific medical conditions rather than a generic one-size-fits-all model.

Various benefits include consolidating and organizing the underlying data in manner that makes information easily understandable and accessible to patients and health care providers, enabling targeted preventive medicine tailored to individual patients in an effective and efficient manner, and facilitating large scale studies, and promoting the active engagement of the patient in local, regional and national research.

As discussed above, one main reason why current PHRs have not yet been able to deliver the benefits articulated in the 2025 ONC vision is likely to be ease of use. Despite the fact that the user-friendly nature of a software application is often associated with the personalization level of the application, most current PHRs have adopted a one-size-fit-all model. The tailor-made, on-demand experience that the community is now expecting from other online services (e.g., on-demand TV, personalized marketing and personalized banking) should be reflected in the services provided by the PHR.

Furthermore, it has been found that the adoption of a condition-driven health data model rather than the widely used event-based and institution-based data model may improve widespread adoption. One of the drawbacks of conventional event-based data model is that, when reviewing, for instance, a patient's diabetic condition within current PHRs, clinicians must find and navigate to measurements, appointments, medications and procedures among a potentially large set of data points over time. The condition-driven model allows the individualized PHR to be organized around a) the conditions of concern to each individual, b) the potential correlation between these conditions, and c) the potential relation between the manifestations of these specific conditions in the PHRs of the patient's relatives.

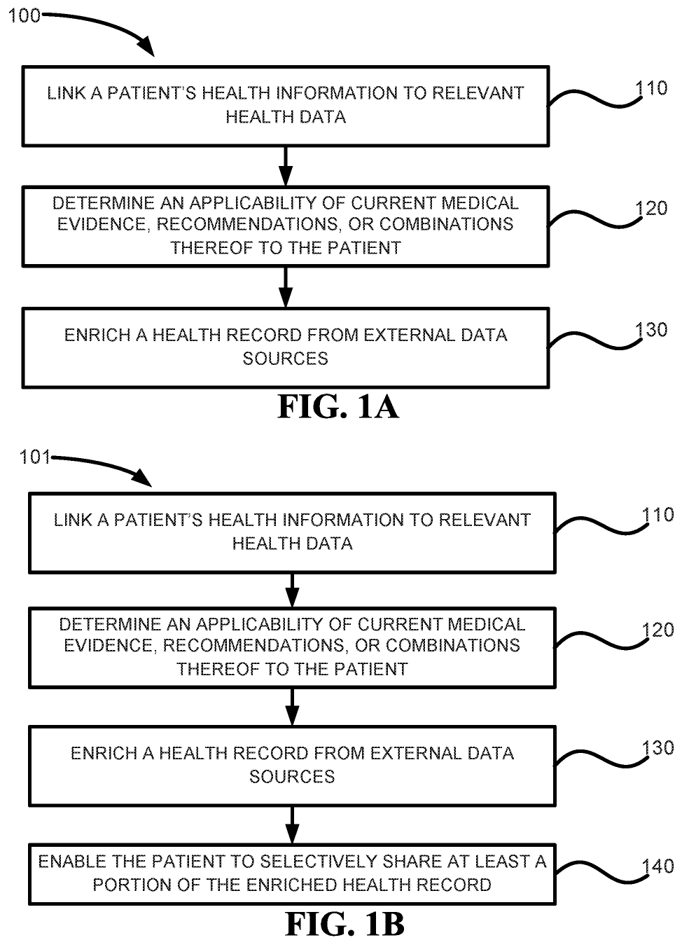

FIGS. 1A and 1B illustrate various methods for providing a health record architecture according to various embodiments. FIG. 1A illustrates method 100 for providing a health record architecture, which may include linking a patient's health information to relevant health data (step 110), automatically determining an applicability of current medical evidence, recommendations, or combinations thereof to the patient (step 120), and enriching a health record from external data sources (step 130).

Similar to method 100 shown in FIG. 1A, method 101 shown in FIG. 1B may include step 110, step 120, and step 130 as previously described. Furthermore, method 101 may further include enabling the patient to selectively share at least a portion of the enriched health record with a health care provider (step 140).

The enriching of a health record from external data sources, such as one or more electronic health records (EHRs), is not particularly limited and may include consolidating the health record. For example, the health record may be consolidated based on a health condition, which can include physical conditions, mental conditions, and combinations thereof. Thus, exemplary health conditions can include various diseases, such as cardiovascular disease, sexually transmitted diseases, cancer, mental disorders, and combinations thereof.

In some embodiments, the consolidation of the health record may include reducing redundancies. For example, in some embodiments, the consolidation of the health record may include creating a health care record map. Health care record maps are not particularly limited and, in various embodiments, may include linking the records extracted from one or more original health care records contained within and EHR system or as a result of data generated by a user.

In some embodiments, the health care record map may include semantic mapping. Organizing health data around conditions in various embodiments may allow for a more concise representation of the pertinent health information and may facilitate better access to a complete disease condition view for each person or patient. This representation may be achieved by semantically mapping records from standard event-based EHRs to the various condition-based Personalized Patient Health Records (P2HRs), using the methods and systems disclosed herein.

In various embodiments, the mapping may establish a relation between each event and the medical condition identified in the diagnosis (e.g., arrhythmia). In various embodiments, a scalable network of disease-specific ontologies may be used in order to perform this type of disambiguation as well as support the required mapping between the source and target data models. Exemplary networks may include various categories, such as for cardiovascular diseases: hypertension, coronary artery disease, arrhythmia, congestive heart failure and combinations thereof.

In various embodiments, the underlying semantic inferences between the entities in the source and target knowledge bases may be derived from the Unified Medical Language System (UMLS). UMLS is a Metathesaurus created by the National Library of Medicine, which integrates several standards including HL7, LOIC, and RxNORM into a unified Metathesaurus. There are two additional components in UMLS that may be used support the various embodiments of semantic mapping. The first is a network which categorizes the entities in the Metathesaurus and establishes the relationships among them and the second component is a specialist lexicon which can correctly interpret user-entry errors.

It has been found that in using ontologies to capture knowledge and derive semantic entailment indicates that these ontologies can quickly grow and become hard to manage and update. Thus, in some instances, the updating of the ontology can become increasingly critical in a field where new discoveries are made on continuous basis. Therefore, some embodiments may allow for the defining of the mapping between UMLS and the P2HR knowledge base through an ontology-to-ontology language. Such embodiments may help address disambiguation and may also facilitate inferences based on, for example, ranges of values of test results, symptoms, and combined predictors.

The ability to make these types of inferences may facilitate correctly associating a predictor (e.g., vital signs, drugs, or procedure) with a specific disease condition. For example, the result of a test (e.g., blood pressure) can be attributed to two or more disease conditions. Also, anticoagulants can also be prescribed for a number of diseases including heart diseases.

Establishing the mapping using an ontology-to-ontology language may allow for a sustained long term management system and methods that allow for the updating of the knowledge base. Furthermore, the level of usability and accuracy of the resulting knowledge base may be updated. Towards meeting this latter requirement, a data driven approach may be used. For example, in some embodiments, network of disease ontologies for cardiovascular diseases and associated mapping may be developed using data extracted from the local health information exchange, such as for a region segment of the population.

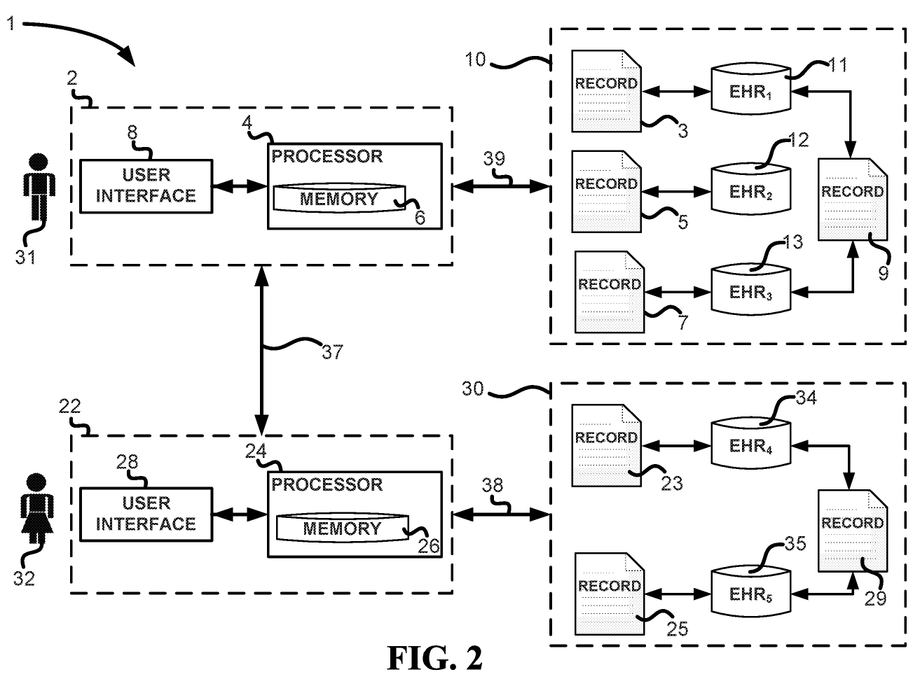

FIG. 2 illustrates exemplary health record architecture system 1 according to various embodiments. System 2 may include a user interface 8, such as a display, a processor 4 in communication with an electronic health record databases 11, 12, and 13, and a non-transitory, computer readable storage medium 6. In various embodiments, the non-transitory, computer readable storage medium 6 may have instructions stored therein that cause the processor 4 to extract and link a patient's health information from an electronic health record, (e.g., record 3, record 5, record 7, and record 9) to relevant health data, automatically determine the applicability of current medical evidence, recommendations, or combinations thereof to the patient, and enrich the health record.

In various embodiments, the electronic health records (e.g., record 3, record 5, record 7, and record 9) may be stored on one or more electronic health record databases (e.g., electronic health record database 11, electronic health record database 12, and electronic health record database 13). In various embodiments, the group of databases 10 may be in electrical communication with system 2, such as with information link 39.

In various embodiments, user 31 of system 2 may allow or authorize an information exchange with another user, such as user 32. In various embodiments, user 31 and user 32 can authorize an information exchange, such as information exchange 37. Thus, the instructions, which when executed by the processor, may cause the processor 4 and/or 24 to deliver at least a portion of the enriched health record after approval from the patient within the architecture to a third-party.

Information exchanges are not particularly limited and may include exchanges over a network, such as a peer-to-peer network. Thus, user 32 of system 22 may authorize system 22 via user interface 28, processor 24 and memory 26 to share one or more of health record 23 and health record 25 with user 31. In various embodiments, groups of electronic health record databases (such as databases 11, 12, 13, 34, and 35) may be configured to share one or more health records. For example, as shown in FIG. 2 group of databases 10 and group of databases 30 may be configured to share records (e.g., record 9 and 29 respectively) within their respective or individual groups (e.g., between EHR₁ 11 and EHR₃ 13 for group of databases 10 and EHR₄ 34 and EHR₅ 35 for group of databases 30).

In various embodiments, the health record architecture systems may be configured to create a health care record map, such as maps including semantic mapping as previously described. In various embodiments, the health care record map may include links to the original health care records contained within an electronic health record (EHR) systems. Furthermore, in some embodiments, the system may be configured to consolidate the health record, for example, automatically.

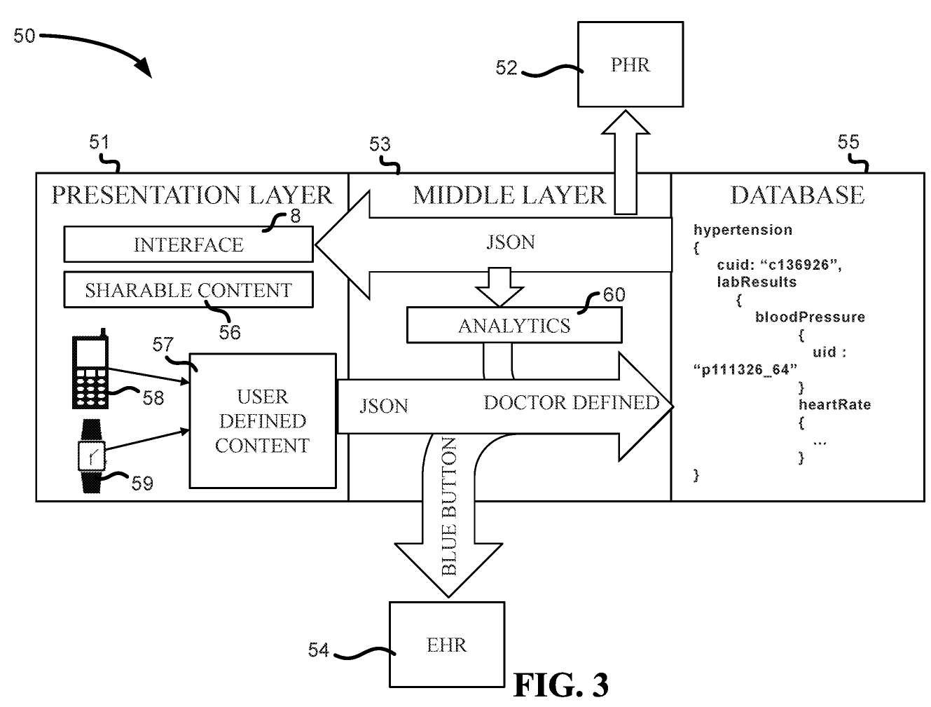

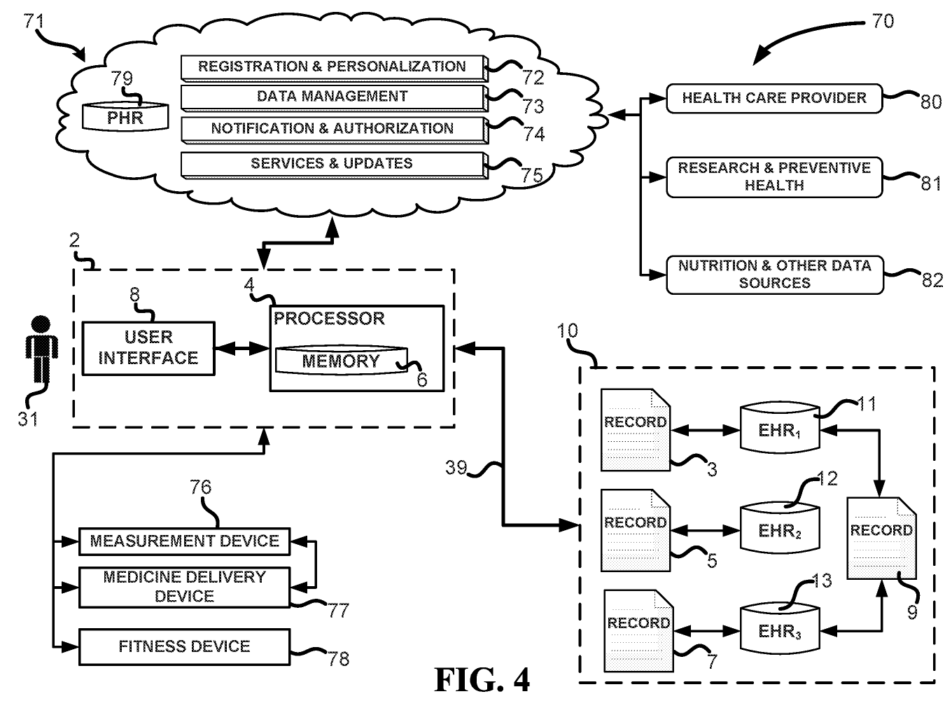

FIG. 3 illustrates a three tier health record architecture according to various embodiments. Three tier system 50 may include a presentation layer 51, a middle layer 53, and a database 55. Presentation layer 51 may include user interface 8, sharable content 56, and user defined content 57. In various embodiments, user defined content 57 may include content from various devices that may measure and/or store health data, such as a mobile phone 58 or smart watch 59. In some embodiments and with temporary reference to FIG. 4, the device may be a health measurement device 76 (e.g., a blood sugar monitor), a medicine delivery device 77 (e.g., an insulin device), or a fitness device 78. Exemplary fitness devices 78 may include an activity tracker, such as a FITBIT®, a registered mark of the Fitbit, Inc. a registered corporation of Delaware.

In various embodiments, the user defined content may interact with the database 55, for example, with clinical document architectures (CDAs), such as Blue Button-Extensible Markup Language (XML), Fast Healthcare Interoperability Resources XML (FHIR-XML), and JavaScript Object Notation (JSON). Furthermore, in various embodiments, the user defined content 57 may be used to enrich the person health record (PHR) 52.

In various embodiments, the middle layer 53 (or middle tier) may manage information flow (e.g., with analytics 60) between the presentation layer and the data management layer (database 55). The middle layer 53 may be configured to handle the mapping from the event-based XML files of the EHRs to the condition-based JSON files. In addition, JSON files retrieved from other health care providers or external devices are also mapped into the condition-based data model as shown in FIG. 3. In various embodiments, the mapping may be done manually by the user, in other embodiments, this mapping may be automated. The mapping may begin when the user adds a new condition. The interface 8 may then prompt the user to establish the relationships between the extracted files and the given condition. The middle tier 53 may also be responsible for connecting a given person's P2HR to the P2HRs of other individuals belonging to the person's sub-network. The sub-network may be defined by the user and may identify the other peers from the general network that the user would like to communicate with.

The database 55 or data management tier may be responsible for storing and retrieving information. In various embodiments, each document in the database may include information related to a given disease or condition. Thus, in various embodiments, information may be passed between the three tiers (presentation layer 51, middle tier 53 and database layer 55), for example, by using a JSON string format.

FIG. 4 shows the general framework of an exemplary PHR framework according to various embodiments. PHR framework 70 may include various modules, such as a personalization and registration module 72, data management module 73, services and updates module 75, and a notification and authorization module 74.

### Personalization and Registration Module

In various embodiments, the concept of personalizing the PHR by using a condition-based model may be sustained throughout the life-cycle of the PHR. When patients initially register, the personalization and registration module 72 may dynamically and interactively create a set of questions enabling the rapid capture of the patient's health status. For instance, knowledge of the gender of the patient will eliminate, in either case, a large number of gender-specific questions. Similarly, entry of the date of birth will allow the registration to be tailored to health conditions pertinent to the appropriate age group. In addition to its efficiency, this approach offers the possibility to drill down and gather information for important health conditions comprising historical information related to a given disease, family members suffering from the same disease, and previous and current health care providers related to this condition.

In various embodiments, the interactive and dynamic registration questionnaire may be the first step of the personalization process. Thus, in some embodiments, the next step may include generating a personalized PHR that will organize the health information provided both by the patient as well as that obtained through the health care provider, for example, in a semantically meaningful way.

### Data Management

In various embodiments, data models may be designed around basic primitive objects (e.g., documents in MongoDB technical terms). These primitive objects may include basic information, measurement, procedure, lab test, prescription, and relation. The primitive objects may be subject to update and extension without incurring any disruption to the operation of the application, in some embodiments.

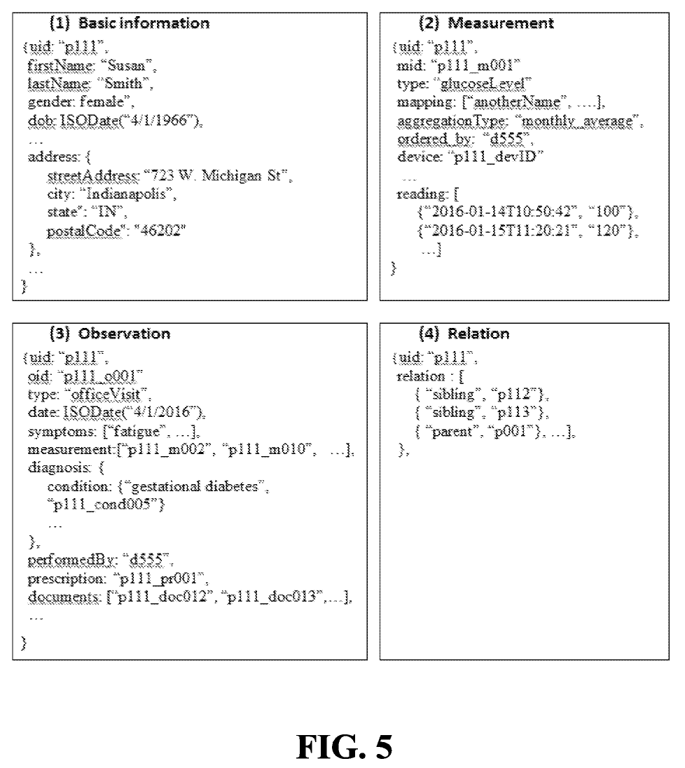

FIG. 5 shows a sample instantiation of four of the primitive objects, namely basic information, measurement, observation and relation. These primitive objects may be patient-specific and generated by the registration and personalization module 72, for example, as shown in FIG. 4. All of the objects reference a “uid” or unique health identification, which is a unique health id for the patient.

In various embodiments, the health care provider may also be associated with a unique identifier, shown in FIG. 5, with the existing National Provider Identifier being the most logical choice. Moreover, all measurements, observations and scanned documents may also have a unique identifier. These latter identifiers can be constructed by prepending the uid of the patient to a unique sequence for each element type.

Various embodiments may include one or more of the following features of the primitive objects:
The measurement type includes a potential list of mappings. These mappings are added as desired to each individual patient record in order to support interactions with health care providers that use different vocabularies. Mapping between different vocabularies can be performed by using a meta-thesaurus such as the UMLS.
The primitive measurement object includes an aggregation type field which indicates how the data should be aggregated (e.g., sampling, monthly averages, cumulative averages, etc.). The aggregation type allows the definition of the most appropriate summative method for high velocity data. In various embodiments, methods may be customizable to each individual, condition and measurement. For instance, different conditions in the same patient may rely on different aggregations of weight or blood pressure readings.
The reading sequence may be a sequence of tuples including a timestamp and a value. The sequence may be updated with every reading associated with a measurement. Furthermore, a new measurement object may be instantiated if any of the underlying fields such as the ordering party or the device uid are changed.
The observation object is used to capture the results of an encounter between a patient and a health care provider. Depending on the result of the observation, the PHR may be restructured to highlight a new condition or the observation may be linked to an already existing health condition.
In some embodiments, the relation object will help identify the type of relation (e.g., parent, sibling, child, etc.) as well as the associated uid of the relative. The underlying application will then perform regular updates based on routine updates to the relative's PHR or based on newly discovered research to establish the association among any of the individual's health conditions and those of a relative. Furthermore, a scoring mechanism may be established and dynamically refined to indicate the strength of this association with each of the relatives and for each of the specific health conditions.

### Services and Updates

In some embodiments, each individual or user may interact with the server and the health care provider via the user interface 8, such as through a personal digital assistant (PDA). Both the server application and the PDA-application may have a three-tier architecture: 1) the front-end is implemented using HTML, CSS and JavaScript, 2) the middle layer is based on GoLang, and 3) the back-end uses Mongo-DB. On the server, Mongo-DB may be used to store all the data related to the registration, personalization, authentication, services and updates.

In some embodiments, there may be two main types of profiles: the patient profile and the health provider profile. The health provider profile may classify the health providers into roles including physician, nurse, insurance, institution, etc. The physician profile may allow the user to interact with the patient through messaging and enables the physician to organize the patients into different groups (e.g., a “critical watch list”). The physician, once authorized by the patient, can also place the patient's measurements under alert. This configurable functionality may enable the physician to receive an alert, for example, when the glucose level of the patient exceeds a certain level.

One of the functionalities of the services and updates module 75 shown in FIG. 4 is to extract health information from different sources of specific interest to the patient. This information can be the result of information from a health care provider 80, a nutrition database 82 or preventive health information 81. This functionality may help the timely translation of scientific findings into immediate benefit to the patient thereby promoting retention and mainstream engagement.

### The Notification and Access Authorization Module

As previously mentioned the structure of the communication of the framework may include the combination of various layers. The first layer may include a cloud server 71 and supports the communication between the patient and the community. This layer may have a centralized architecture and may include the notification and access authorization module 74 as shown in FIG. 4. In various embodiments, the notification and access authorization module 74 may be responsible for various tasks, such as:
The authentication of all users, including patients, service providers and researchers;
The management of patient-health care provider access authorizations which are given on a per condition basis by the patient to the health care provider. Connection rules are managed through the front-end and data access rules are managed through the back-end in MongoDB; and
The management of access authorizations required for the participation of the patient in research studies.

The second communication layer may include a Peer-to-Peer network. In various embodiments, the peers may be autonomous and self-organize in a private sub-network that co-exists with other patient-centric or provider-centric networks. Once a user is authenticated by the notifications and access authorization module 74 in the first layer, they can exchange data directly with their selected peers without the involvement of the server. This mode of communication is used for the exchange of private data between the patient and the health care provider. In addition to scalability and congestion control, the Peer-to-Peer network offers better data exchange security.

### The Personalized PHR

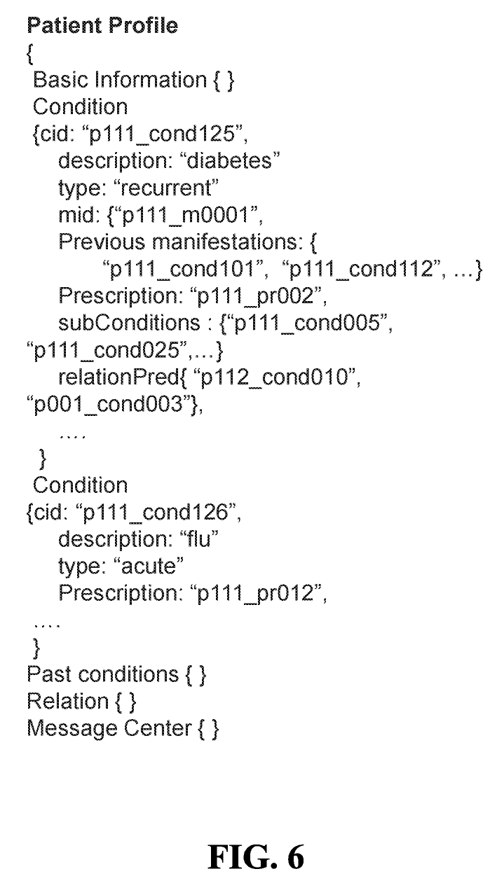

The role of the registration and personalization module 72 is to generate the specific meta-model for the PHR and to populate it with the appropriate data by using the other modules including the services and updates module 75 mentioned above. FIG. 6 shows an exemplary high level representation of this model for an exemplary individual. The skeleton of the model was generated by the registration and personalization module 72, as previously described, when the patient first interacts with the system. This model may then be enhanced and updated as it is enriched by the user, the health care provider, and the users' network.

The services and updates module 72 may be configured to rely on available application program interfaces (APIs) in order to extract the information from different EHRs and research data sites. The APIs can be triggered on a regular basis to update the PHR record of each individual patient.

### Data Retrieval and Indexing

Most current database applications either provide ease of access for a specific individual or provide ease of access as across a large set of individuals. For instance, organizations often segregate among the two types of accesses by using a transactional database (e.g., EHR), as well as a data warehouse for analysis and reporting purposes. One of the emergent applications of this combination is social computing. The social media model is relatively simple and based on relations such as “is a friend” and/or “is a follower”. In various embodiments, the PHR model may support both transaction-based interactions (e.g., health care provider—patient) as well as analytics-based processing for large scale studies. However, the health context makes the data retrieval and management complex. Therefore, in some embodiments, communication has been segregated into two layers, services have been carefully assigned to the client or the server and data management has been associated with the most appropriate application depending on the context.

Patient local data may be managed by the client application and then bi-directionally synchronized with the server as desired. In some instances, for high velocity data, aggregation can help reduce the size of the data that needs to be stored on the PDA.

Thus, the exemplary embodiments disclosed herein may help close important gaps in the health care research, clinical practice and patient continuum by allowing patients to better manage their health, and benefit from up-to-date research efficiently and effectively. Exemplary systems, architecture, and methods disclose herein may also link a patient's health information to relevant health data about their family members, automatically determine the applicability of current medical evidence or recommendations to the patient, and enable the patient to enrich his record from external data sources. These features will facilitate mainstream adoption and, more importantly, sustained engagement by the patient in managing his health.

Thus, various aspects disclosed herein include developing methods to custom-build on-demand applications that will manage data for each individual patient in a personalized and focused manner; and identifying mechanisms that can translate research findings into practical and systematic methods for organizing patient health information.

This level of quality of service will keep patients engaged and interested in the management of their data. Furthermore, it will encourage patients to become willing participants in large-scale medical studies that can further improve research. Thus, the present disclosure leverages the viral effect of social networks towards a customizable solution for patient-centered health record and a cost-effective mechanism to provide answers to important questions that are unattainable through traditional research studies.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations that fall within the scope of the claims, together with all equivalents thereof.

What is claimed is:

1. A method for providing a health record architecture, comprising:
linking a patient's health information to relevant health data;
automatically determining an applicability of current medical evidence, recommendations, or combinations thereof to the patient; and
enriching a health record from external data sources.

2. The method of claim 1, wherein the enriching of the health record comprises consolidating the health record.

3. The method of claim 2, wherein the health record is consolidated based on a health condition.

4. The method of claim 3, wherein the health condition is a disease.

5. The method of claim 2, wherein the consolidation of the health record comprises reducing redundancies.

6. The method of claim 2, wherein the consolidation of the health record comprises creating a health care record map.

7. The method of claim 6, wherein the health care record map comprises links to records extracted from the original health care record contained within an electronic health record (EHR) system or as a result of data generated by the user.

8. The method of claim 6, wherein the health care record map comprises semantic mapping.

9. The method of claim 1, further comprising enabling the patient to selectively share at least a portion of the enriched health record with a health care provider.

10. The method of claim 1, wherein the enriching of the health record from external data sources is based on a condition of the patient.

11. The method of claim 1, wherein the enriching of the health record from external data sources is based on a condition of a group of patients.

12. The method of claim 1, further comprising delivering of a portion of the enriched health record of the patient to a third-party after approval from the patient.

13. A health record architecture system comprising:
a display;
a processor in communication with an electronic health record database and a non-transitory, computer readable storage medium;
the non-transitory, computer readable storage medium having instructions stored therein that cause the processor to:
extract and link a patient's health information from an electronic health record to relevant health data;
automatically determine the applicability of current medical evidence, recommendations, or combinations thereof to the patient; and
enrich the health record.

14. The health record architecture system of claim 13, wherein the instructions, which when executed by the processor, are configured to receive updated data from a personal health measurement device.

15. The health record architecture system of claim 13, wherein the instructions, which when executed by the processor, are configured to create a health care record map.

16. The health record architecture system of claim 15, wherein the health care record map includes semantic mapping.

17. The health record architecture system of claim 15, wherein the health care record map comprises links to original health care records contained within an electronic health record (EHR) system.

18. The health record architecture system of claim 13, wherein the instructions, which when executed by the processor, are configured to cause the processor to consolidate the health record.

19. The health record architecture system of claim 13, wherein the instructions, which when executed by the processor, are configured to cause the processor to deliver at least a portion of the enriched health record after approval from the patient within the architecture to a third-party.

20. The health record architecture system of claim 13, wherein the processor communicates with other user-owned devices using a Peer-to-Peer network.

21. A non-transitory computer readable storage medium bearing instructions for providing a health record architecture, the instructions, when executed by a processor in electrical communication with an electronic health care database, cause the processor to perform operations comprising:
linking a patient's health information to relevant health data;
automatically determining an applicability of current medical evidence, recommendations, or combinations thereof to the patient; and
enriching a health record from the electronic health care database.
